(19) United States
(12) Patent Application Publication
(10) Pub. No.: US 2013/0026137 A1
(43) Pub. Date: Jan. 31, 2013

(54) DEVICE AND METHOD FOR GENERATING A PULSED ANISOTHERMAL ATMOSPHERIC PRESSURE PLASMA

(71) Applicants: Eckhard Kindel, Greifswald (DE); Klaus-Dieter Weltmann, Binz (DE); Norbert Lembke, Greifswald (DE); Thomas Kocher, Neuenkirchen (DE)

(72) Inventors: Eckhard Kindel, Greifswald (DE); Klaus-Dieter Weltmann, Binz (DE); Norbert Lembke, Greifswald (DE); Thomas Kocher, Neuenkirchen (DE)

(73) Assignee: Leibniz-Institut fuer Plasmaforschung und Technologie e.V., Greifswald (DE)

(21) Appl. No.: 13/512,261

(22) PCT Filed: Nov. 27, 2010
(86) PCT No.: PCT/DE10/01390

§ 371 Date: Sep. 10, 2012

(30) Foreign Application Priority Data
Nov. 27, 2009 (DE) 10 2009 047 220.7

(51) Int. Cl. H01J 7/00; B08B 7/00; C23C 16/50; B44C 1/22; A61L 2/14; H05H 1/24

(52) U.S. Cl. 216/67; 422/29; 313/231.31; 315/111.31; 427/569; 134/1.1

(57) ABSTRACT

The invention relates to a device and a method for generating a pulsed (intermittent), cold, atmospheric pressure plasma, preferably a thread, for precise antimicrobial plasma treatment (antisepsis, disinfection, sterilization, decontamination) of very small surfaces and cavities, including on living human and animal bodies, preferably in the field of medicine, by means of a negative direct-current corona discharge, the device comprising at least one electrode for generating high field strengths, through or around which electrode the gas to be ionized flows in a gas channel, wherein the electrically conductive structure (surface, cavity) to be treated is used as the counter-electrode. Said plasma can also be used in general for cleaning, coating, activating, and etching surfaces.

The invention relates to a device and to a method for generating a pulsed (intermittent), cold, atmospheric-pressure plasma, preferably a filament, for pinpoint antimicrobial plasma treatment (antisepsis, disinfection, sterilization, decontamination) of minute areas and cavities, even of live human and animal bodies, preferably in the field of medicine, by means of a negative DC corona discharge with at least one electrode for generating high field strengths, through or around which the gas to be ionized flows in a gas channel, while the electrically conductive structure (surface, cavity) to be treated acts as the counter-electrode. This plasma may also be used in general for cleaning, coating, activating and etching of surfaces.

### PRIOR ART

Anisothermal plasmas at atmospheric pressure have already been used for many years for treatment of surfaces for the purpose of surface activation, etching, polymerization, film deposition, cleaning and microbial reduction.

For this purpose there have been developed several plasma arrangements, which function, for example, on the basis of a dielectrically hindered discharge [M. Laroussi, IEEE Trans Plasma Sci 24 (1996), 1188-1191], of an arc discharge [DE 19532412 C2], of a corona discharge [M. Laroussi et al., IEEE Trans Plasma Sci 28 (2000), 184-188], or of an HF-excited or microwave-excited jet [H W Hellmann et al., Phys Plasma 6 (1999), 2284-2289, B J Park et al., Phys Plasma 10 (2003), 4539-4544]. These anisothermal plasmas are capable of enhancing chemical and biochemical reactions without a substantial rise in the gas temperature. The electrons in these plasmas have much higher temperature than the heavy particles (ions, neutral particles) and consequently cause excitation, ionization and dissociation when they collide with the atoms and molecules of the process gas. Reactive, neutral and also charged particles formed as a result then react with the surface to be treated.

Because of its antimicrobial efficacy [R. Brandenburg et al., Contrib. Plasma Phys 47 (2007), 72-79] and the capability of generating cold plasmas (room temperature), these plasmas have recently become particularly attractive in the field of medicine (dental medicine) and biomedicine for treatment of thermolabile objects such as biological cells and tissues [I. E. Kieft et al., IEEE Trans Plasma Sci 33 (2005) 771-775]. In order that the temperature of the heavy particles, in other words the base gas, can be kept low, pulsed plasmas with very short current pulses in the ns range and repetition rates in the kHz range are generated, so that the mean energy input remains low.

In the paper of R. E. J. Sladek et al. in IEEE Trans Plasma Sci 32 (2004) 1540-1543, a “plasma needle” for treatment of dental caries is described. The plasma formed at the tip of a wire ground to 0.3 mm thickness has an extent of only 1 mm, making it difficult to achieve effective (efficient) antimicrobial decontamination, for example in cavities (root canal, gingival sulcus) in the mouth. The HF generator (13.56 MHz) used for generating the plasma, together with a corresponding matchbox, makes the entire system relatively expensive.

In the paper of C. Jiang et al. in Plasma Process. Polym. 6 (2009), 479-483, a “dental probe” for disinfecting the root canal of an extracted tooth is described. The helium/oxygen jet, which has a length of 2.5 cm, is generated by means of a high-voltage pulse generator (6 kV, pulse width 100 ns, pulse repeat rate 1 kHz) in a hollow electrode arrangement. By means of SEM (scanning electron microscopy) it is demonstrated that this cold plasma 35° C.) is capable of achieving disinfection (destroying biofilm) in the root canal. In this case also the use of an expensive generator is necessary.

In GB Patent 2246955A, a device for killing microorganisms is described. By means of a DC high-voltage supply (10 kV), a negative corona discharge with limited current (100 μA) is generated in air at a probe consisting of a pointed electrode. The negative air ions formed therein are then supposed to achieve destruction of the microorganisms.

The probe is connected via an electrical line (wire) to the negative pole of the DC voltage source. The positive pole is either grounded and/or connected to the patient to be treated. The extent of the plasma generated at the tip is smaller than one millimeter.

During treatment, the physician guides the probe to the site to be decontaminated as far as a preferred distance of 6 mm.

On the basis of this patent, the firm of DENTRON has marketed a device by the name of “Biogun”. According to the firm, the disinfecting effect is supposedly achieved by the superoxide anion radical O₂⁻. The corona discharge generated at the tip most likely brings about generation of an ion wind (which is known from physics), which transports the radical to the desired site.

Since the discharge is generated in air, it is accompanied by simultaneous formation of ozone, which must then be scavenged by an aspirator, for example in patients with bronchial diseases, thus posing a barrier to simple treatment. Also, the very small extent of the plasma does not permit the direct contact with the contaminated surface that would achieve a much more effective disinfection effect.

In the cited GB 2246955A publication, the negative DC corona discharge used is generated in a stationary air environment. The electrode is located directly in air and not in a gas channel provided on the outside thereof, thus also explaining the small extent of the corona.

Other publications belonging to the prior art are DE 102008008614 A1 and WO 2009/101143 A1. These describe methods and devices for treatment of living cells by means of a cold atmospheric pressure plasma with simultaneous selective electroporation of the cells for local, selective killing of cancer cells, improved wound treatment and a better antimicrobial plasma effect.

In the field of medicine, special, HF-excited plasmas have been used for many years for coagulation (argon plasma coagulation: U.S. Pat. No. 4,060,088 A, U.S. Pat. No. 4,781,175 A, DE 102008004843 A, EP 1148770 A) or for high-frequency surgery. The alternating current generated by a high-frequency generator is conducted with high frequency via the resulting plasma through the human body, in order to destroy and cut tissue selectively and at the same time achieve hemostasis by occlusion of the affected vessels. The high-frequency powers needed for this purpose are in the range between 50 W (dental or ophthalmological surgery) and at most 400 W. Because of the high energy input needed for these processes, these devices are therefore not suitable for decontamination of thermolabile surfaces.

The disadvantage of the solutions described in the prior art consists not only in the fact that the plasmas have a very small extent but also in the fact that expensive generators are necessary and excessively high plasma temperatures, unsuitable for thermolabile surfaces, are generated. Furthermore, the current generated by the plasma and passing through the body of the patient is so high that is causes nerve stimulation (faradization) and produces harmful substances, and so it cannot be medically applied without special precautions (narcosis, scavenging of the harmful substances).

### OBJECT OF THE INVENTION

The object of the invention is to eliminate the disadvantages of the solutions cited in the prior art.

### ACHIEVEMENT OF THE OBJECT

The object is achieved in accordance with the features of the claims.

According to the invention, there is provided a simple, inexpensive and handy device for generating a pulsed, cold, filament-like (micro) atmospheric-pressure plasma for pinpoint modification (antimicrobial decontamination) of minute areas and cavities, even in live human and animal bodies, which device does not cause any irritations (and is therefore gentle) and is of simple construction.

This is achieved by generation of a negative DC corona discharge with a simple DC high-voltage supply and with at least one electrode for generating high field strengths, preferably in the range of 5 kV/cm to 10 kV/cm, through or around which the gas to be ionized flows in a gas channel, while the electrically conductive object to be treated (surface, cavity, human, animal, plant) functions as the counter-electrode.

By appropriate choice of parameters, such as gas type, gas flowrate, amplitude of the supply voltage, a pulsed plasma filament with a diameter of approximately 30 μm and a length of 1 cm is generated. When argon is used as process gas with a flowrate of 0.5 slm together with a DC voltage of 10 kV to 14 kV, pulsed currents of 400 mA to 1.4 A with widths at half height of 20 ns and a repetition rate of 1 to 3 kHz are generated. This would then correspond to mean powers of 0.16 W to 0.56 W with mean currents of 16 μA to 40 μA. At these low powers or currents, no or only slight heating and no nerve stimulation (faradization) occurs during application to the human body. When noble gases are used, the generated ozone concentration is minimal (lower by a factor of at least two to three compared with the former MAK value [maximum exposure level] of 0.1 ppm).

Because the length of the plasma is approximately 1 cm, the user is able to bring the plasma directly into contact with the contaminated object. The length of the plasma is determined mainly by the gas flowrate and by the amplitude of the applied high voltage.

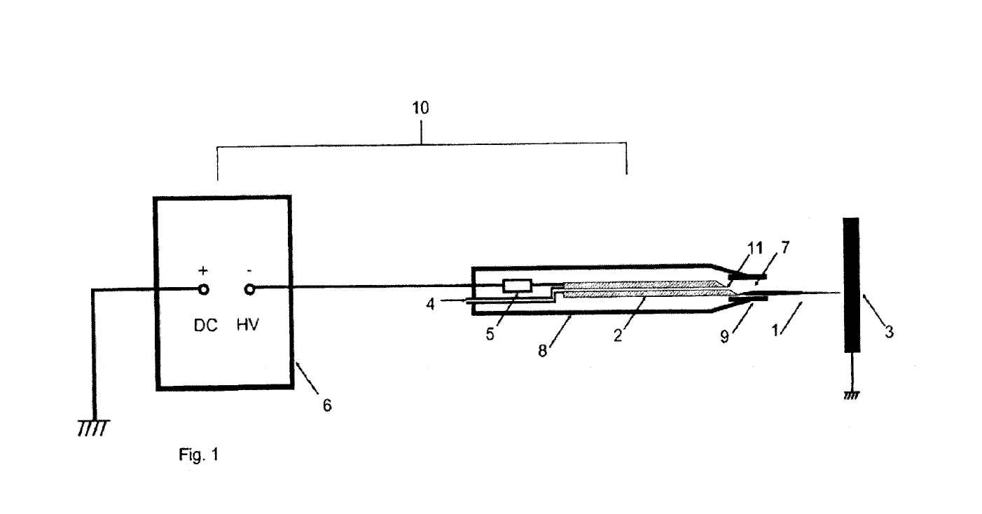

The invention will be explained in more detail hereinafter on the basis of FIGS. 1 to 3, without limiting the invention to these examples.

### EXEMPLARY EMBODIMENTS

The invention will be explained in more detail with the drawings illustrated hereinafter in FIG. 1 to FIG. 3. The following reference numerals will be used to denote the individual elements:

| | | | |
|---|---|---|---|
| 1 | Plasma | 2 | High-voltage electrode |
| 3 | Grounded electrode | 4 | Process gas |
| 5 | High-resistance resistor | 6 | High-resistance DC voltage supply |
| 7 | Gas channel | 8 | Housing (insulating material) |
| 9 | Capillary | 10 | Device |
| 11 | Gas channel of the high- | | |
| | resistance electrode | | |

FIG. 1 schematically shows the basic structure of the device. Inside a housing (8) of non-conductive material, a current-limiting resistor (5) and a high-voltage electrode (2) similar to an injection cannula of an injection syringe are disposed in such a way in a gas channel (7) that process gas (4) flows through gas bore (11) of electrode (2). Resistor (5) is connected to the negative pole of a high-resistance DC voltage supply (6). The positive pole is grounded, as is electrode (3). At sufficiently high voltage, an intermittent plasma filament, directed toward grounded electrode (3), is generated at the tip of electrode (2). Capillary (9), consisting of a heat-resisting material, forms gas channel (7).

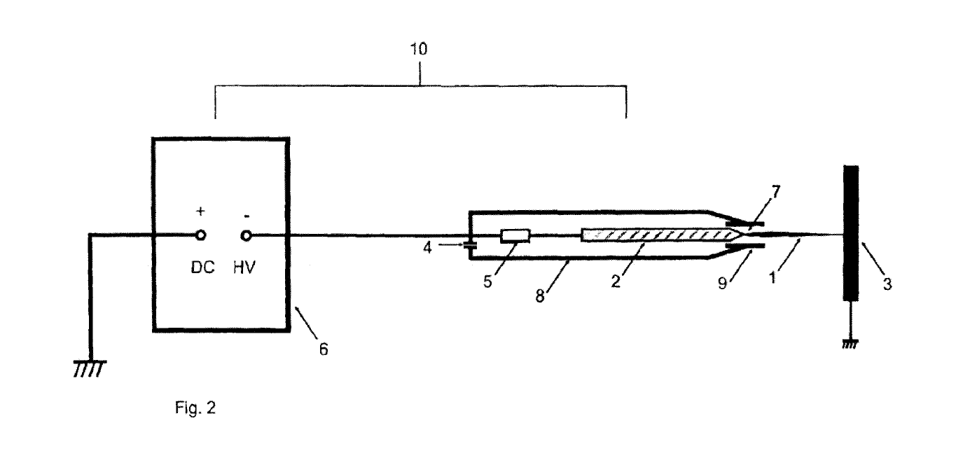

FIG. 2 shows a similar arrangement. In this case high-voltage electrode (2) has the form of a needle, around which process gas (4) flows.

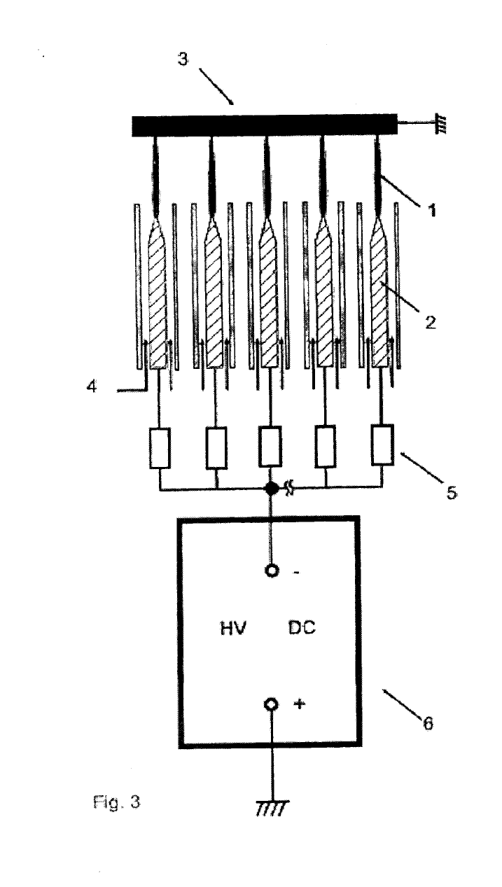

Upscaling is also possible by connecting a plurality of electrodes in parallel, each electrode having a current-limiting resistor (FIG. 3).

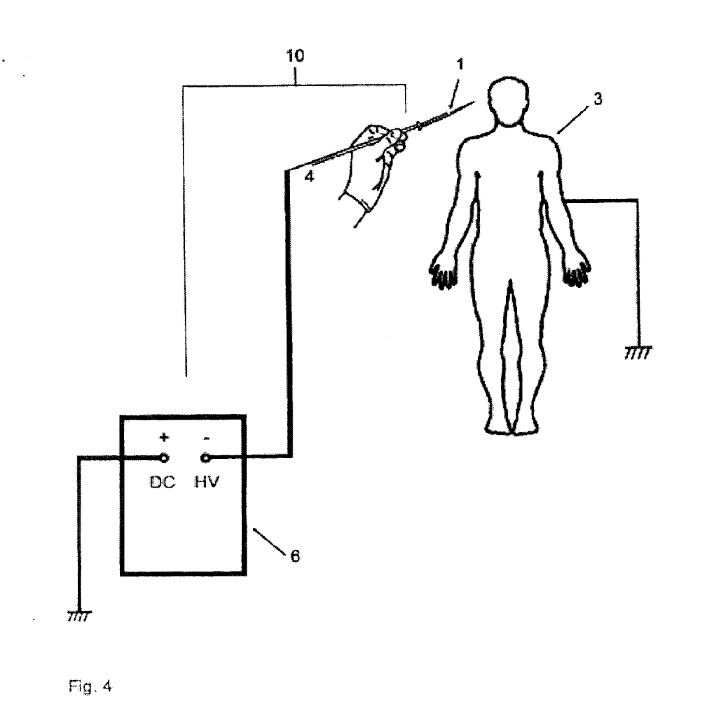

By virtue of the very low discharge currents (≦50 μA), this plasma may also be used directly for cosmetic or medical purposes on humans or animals (FIG. 4).

To prevent charge accumulations, both the user and the test subject must then be grounded.

1. A device for generating a cold, pulsed, atmospheric-pressure plasma on an electrically conductive surface by an intermittent, negative DC corona discharge, comprising:
an insulating housing with at least one high-voltage electrode suitable for generating high field strengths, which is disposed in a gas channel and through which the gas to be ionized flows in a gas bore of the high-voltage electrode or around which it flows in a gas channel and with which the negative pole of a DC voltage source is electrically connected, while the positive pole is grounded and the surface to be treated acts as the counter-electrode.

2. The device according to claim 1, wherein the atmospheric-pressure plasma is a plasma filament, which has a length of 1 cm and a diameter of 30 μm.

3. The device according to claim 1, wherein the high-voltage electrode
is disposed in a gas channel surrounding the high-voltage electrode, and
a) is an individual ground electrode with an internal bore, or
b) is an individual needle-shaped electrode, around which the gas flows, or
c) a plurality of such electrodes according to a) or b) is used as the high-voltage electrode.

4. The device according to claim 1, wherein the high-voltage electrode is electrically connected via a high-resistance, current-limiting resistor to a negative pole of the high-resistance DC voltage supply.

5. The device according to claim 1, wherein the high-resistance, current-limiting resistor is part of the high-voltage electrode.

6. The device according to claim 1, wherein noble gases, oxygen, air, nitrogen or any desired mixtures of the cited gases are used as the gas to be ionized.

7. A method for generating a cold, pulsed, plasma filament on an electrically conductive surface, comprising:
discharging an intermittent, negative DC corona with the device according to claim 1,
wherein the length of a plasma filament is controlled by a gas flowrate and by an amplitude of an applied high voltage.

8. The method according to claim 7, wherein, when argon is used as process gas with a flowrate of 0.5 slm together with a DC voltage of 10 kV to 14 kV, pulsed currents of 400 mA to 1.4 A with widths at half height of 20 ns and a repetition rate of 1 to 3 kHz are generated.

9. A method for pinpoint antimicrobial plasma treatment of minute areas or cavities, comprising:
generating a cold, pulsed, atmospheric-pressure plasma on an electrically conductive surface by an intermittent, negative DC corona discharge using the device according to claim 1.

10. A method for modifying, cleaning, coating, activating or etching of a surface, comprising:
generating a cold, pulsed, atmospheric-pressure plasma on an electrically conductive surface by an intermittent, negative DC corona discharge using the device according to claim 1.

11. The method according to claim 9, wherein said pinpoint antimicrobial plasma treatment comprises antisepsis, disinfection, sterilization, and/or decontamination.
